(19) United States
(12) Patent Application Publication
(10) Pub. No.: US 2002/0019356 A1
(43) Pub. Date: Feb. 14, 2002

(54) HYPERBARIC OXIGENATION (HBO) AS THE METHOD FOR ENHANCEMENT EFFICIENCY OF ANTI-HIV SUBSTANCES IN INHIBITION THE HUMAN IMMUNODEFICIENCY VIRUS (HIV) REPRODUCTION AND REDUCTION OF THE CYTOTOXIC EFFECT OF THOSE SUBSTANCES AS WELL

(76) Inventors: VLADIMIR ILICH PAKHOMOV, MOSCOW (RU); EDUARD VLADIMIROVICH KARAMOV, MOSCOW (RU); ALEXANDR EVGENEVICH SOKOLOV, MOSCOW (RU); GALINA VLADIMIROVNA KORNILAEVA, MOSCOW (RU); MIKHAIL YUREVICH SHTSHELKANOV, EKATERINBURG (RU); VLADIMIR NIKOLAEVICH KOSTYUNIN, MOSCOW (RU)

Correspondence Address: ROTHWELL FIGG, ERNST & KURZ P C, 555 13TH STREET N W SUITE 701-E, WASHINGTON, DC, 20004

(21) Appl. No.: 09/303,652

(22) Filed: May 3, 1999

(30) Foreign Application Priority Data
May 18, 1998 (RU) 98109354

(51) Int. Cl. C07H 19/00; C07H 19/048; A61K 31/70; A61G 10/00; A01N 43/04; A61K 31/04; A01N 33/18; A01N 33/24

(52) U.S. Cl. 514/43; 514/42; 514/740; 536/28.1; 536/28.2; 128/202.12

(57) ABSTRACT

A method of increasing efficiency and/or decreasing the cytotoxic effect of a human immunodeficiency virus (HIV) reproduction-suppressing drug such as Azidothimidin (AZT) involves administering the drug to an HIV-positive patient and subjecting the patient to hyperbaric oxygenation (HBO).

### BACKGROUND OF THE INVENTION

The present invention relates to the methods of prophylactics and therapeutics of Human Immunodeficiency Virus (HIV) infection well as to the Acquired Immunodeficiency Syndrome (AIDS), which is etiologically connected therewith.

More specifically this invention permits to increase the Azidothimidin's (AZT) inhibition effect to the HIV reproduction and simultaneously, to decrease the cytotoxic effect.

It is known the way for suppression of reproduction HIV with the help of 3-azido-2,3-didezoxythimidim (Karamov E. V., Lukahev V. V., Gorbacheva A. P. etc. <<Inhibition of reproduction of Human Immunodeficiency Virus in cell's culture with 5-phosfatum 2,3-didezoxynukleozidov>>. Molecular biology, 1992, volume 26, p.201-206).

However, the application AZT for suppression of reproduction HIV restrains by its high toxicity, first of all for marrow cells and cells of central nervous system.

It is known the way of influence on biological objects by hyperbaric oxygen. In medicine this way is used for treatment some diseases (Undersea biomedical research, 1992, vol.17, p. 122-126).

It is known the way of HBO application for decrease cytotoxic effect of medicines such as vinkristin, rubomiczin, metatreksat etc. (Pakhomov A. E. with the co-authors, copyright certificate N^{o}1341761, priority—January 1985. The bulletin of inventions, 1989, N^{o}42, USSR).

The patent of Russian Federation N^{o}2084212 (Pakhomov A. E with the co-authors, the priority—April 1996) contains the description of a way of HBO application for suppression of reproduction HIV and increase in stability of infected and uninfected cells to this virus.

The research is directed to the development of approach methods for the suppression of the reproduction and/or annihilation of HIV in an infected organism without harm to healthy cells and tissues.

### SUMMARY OF THE INVENTION

Subject of the invention is the HBO as the method of the enhancement of the Azidothimidin inhibition efficiency to the Human Immunodeficiency Virus reproduction Another subject of the invention is the HBO as the method of the Azidothimidin cytotoxic effect decrease.

The other subjects of the invention will be clear from the detailed description of this invention.

### DESCRIPTION OF THE INVENTION

In accordance with the present invention, the method of the increase of anti-HIV substances efficiency and the decrease of cytotoxic effect implied, that human cell lines, sensitive to HIV-infection, are subjected to AZT-addition and a single or multiple HBO exposure in an oxygenous pressure chamber before or/and after being infected by HIV.

For checking the influence of HBO there was used a corresponding infected cell culture which was subjected to AZT, but was not subjected to HBO.

For checking the infection development, there was used a corresponding infected cell culture which was subjected neither HBO nor AZT.

Uninfected cell culture, which was subjected to HBO and AZT, was used as control to the main experiment.

And for checking the viability of the cells itself there was used a corresponding cell culture which was not inoculated by HIV and was not subjected to the HBO and AZT.

The further cultivation of mentioned cell cultures was carried out at customary conditions under normal pressure.

In accordance with present invention, the effect of the combination of the AZT and HBO reduces the level of viral antigen expression and the amount of the proviral DNA copies more then single AZT. Besides, it increases the cell viability in a HIV infected cultures.

Particular embodiments of the present invention, which are illustrating but not limiting the essence and content of the invention, are given below.

The monitoring of the HIV-infections was carried out with help of the following methods:

1.) Determination of the total level of viral antigen expression by means of the method of polyclonal immunoenzyme analysis (EIA) [ABBOTT™ ELISA Kit].

2.) Determination of the concentration of the protein HIV p24 in cultural supernatant by means of p24-monoclonal immunoenzyme analysis [AMPAKTM ELISA Kit].

3.) Determination of the average quantity of proviral DNA copies in a cell by means of the quantitative polymerase chain reaction (PCR) method with two internal standards: for the cellular gene HLA-DQa and for the HIV proviral DNA [1].

The first represents a PCR amplification product of the recombinant HLA-DQa with deletion of 40 bp, and the second is a PCR amplification product of the recombinant gene fragment pol HIV-1 with insertion of 80 bp.

The average amount of proviral DNA copies in a cell is determined by the doubled ratio of the standardised quantity of amplification product of the proviral DNA fragment (the gene pol HIV-1) to the standardised amount of amplification product of the cellular gene (HLA-DQa).

The doubling of the above-indicated ratio is necessary because of the double-allelism of the HLA-DQa gene.

The PCR is carried out by means of a standard procedure [1] in 20-25 μl of a reaction medium containing 20 mM TRIS-HCl (pH 8,8; 20° C.); 50 mM KCl; 2.5 mM MgCl₂; 170 mg/ml gelatine; 100 μM of each dATP, dTTP, dCTP, and dGTP; pair of primers 10-20 pM; Taq-polymerase 1.0-1.5 U; the DNA under study and two internal standards in a working culture.

PCR cyclogram: 30 cycles of the operating conditions of (94° C., 1 min)-(55° C., 1 min)-(72° C., 1 min).

The standardisation of the amplification product is obtained by correlating the fluorescence band intensities of the sample and of the corresponding standard on an electroforegram in 2% agarose gel by colouring it with ethydium bromide and irradiating with ultraviolet rays. Another approach to standardisation is based on the comparison of the radioactivity level of the corresponding electrophoregram bands after hybridising the PCR products with the complementary 32P-labeled probes.

For this purpose, to 10 ml of PCR product are added 0.2 pmol of 32P-labeled complementary probes in 6 ml of a solution containing 30 mM NaCl, 6 mM EDTA, 10% glycerine, 0.002% BPB and XC. The mixture is incubated 5 min at 95° C., 15 min at 55° C., where upon the electrophoresis is conducted in a not denaturing 10% polyacrylamid gel. The corresponding electrophoregram bands are cut out, and the radioactivity level is evaluated through the Cerenkov radiation intensities.

4.) Monitoring the viability by means of direct cell calculation in a Goryaev counting chamber after colouring with the excepting vital stain 0.2% trypan blue. For the direct comparison of the amounts of living cells there is used the so-called MTT-method, which is based on the capability of living cells to reduce the yellow water-soluble tetrazole salt MTT (3-(4,5)-dimethylthiazol-2-ye)-2,5-diphenyl-bromide tetrazole) into insoluble intracellular MTT-formazan crystals, whereas dead cells are not capable to do this [2,3].

A concentrated MTT solution of 10 mg/ml is prepared with a phosphate buffer solution at pH 7.4 and brought into a hollow with the cells at a 10-fold dilution rate.

After incubation over 4 hours at 37° C. the supernatant is isolated with help of microdosing tips of a special design (S-tips).

Into the hollow is added 150 ml of DMSO as solvent. The thereupon measurable optical density is within 540 to 590 nm, i.e. in the absorption range of formazan, is direct proportional to the quantity of living cells [4].

The values of 50 per cent effective AZT dose (ED₅₀) were calculated conformably to oxidised and non-oxidised cells subsequently infected by HIV and treated by AZT in various concentrations.

ED₅₀ values were derived according to the linear regression resulting averaged values of the optical density signal V(%) depending on the logarithm of AZT doses (10⁻⁶; 10⁻⁷; . . . 10⁻¹² M).[8]

It was found that only the first order regression is statistically reliable.

Regression lines are determined with the following formula:

V=a(lg AZT−6)+b

where <<a>> and <<b>> are regression coefficients, calculated by mean square root method.

There are particular examples of using the invention, but they do not limit the essence and subject of the invention.

### EXAMPLE 1

### The decrease of Azidothimidin Toxic Effect to Supt1 T-limphoblastoide Line Cells.

18 million Supt1 cells [5] in 60 ml of the growth medium RPMI-1640 with 10% fetal calf serum, 2 mM L-glutamine and 50 mg/ml genthamycin were placed in each of 2 plastic flasks (75 cm³), mark Costar®, USA, one of which was subjected to HBO (Ox+), and the other was used for counterchecks with intact and infected cells without a HBO exposure (Ox−).

The HBO [6] was carried out in the oxygenous pressure chamber BLKS-303M. The pressure chamber was sealed, blown out with oxygen over 7 min till an oxygen concentration of 98-100% was obtained at the outlet of the pressure chamber, whereupon the oxygen pressure in said pressure chamber was increased up to 1.5 ate. The compression rate was 1,077 ate/min and the time period of isocompression was 40 min. After having finished the HBO process, the oxygen pressure was decreased to atmospheric during 6 min.

Over 40 min after a finish of the HBO—procedure in the each well of the 24-wells plate (Nuclon®, Denmark) added 45 μL of a cell suspension (3×10⁵ cell/mL) and 50 μL of a growth substance with a different concentrations of AZT.

After an incubation over 2 hours at 37° C. an oxygenated and non-oxygenated samples were infected with HIV-1_{BRU} (non-resistant to AZT) and HIV-1_{A216} (resistant to AZT),

The infection was conducted according to the following scheme:

After having centrifuged 24-wells plate at 300 g over 10 min, supernatant was taken out the wells by S-tips (besides 50 mL of a cell's sediment), to which was added 30 ml of the virus-containing supernatant of 480 TCID₅₀.

The determination of the TCID₅₀ was carried out by means of the syncytium forming test: to 100 mL of a cell suspension (5*10⁵ cells/ml) arranged in a hollow of a 96-hollow plate (Costar®, USA) were added different dilutions of the virus-containing supernatant, starting with 1:5 and continued with the dilution rate 2.

After cultivation of 72 h at 37° C., 5% CO₂ and 80% moisture content the presence of syncytium was visually observed.

The TCID₅₀ was found in accordance with the Reed and Muench's method by means of a linear logarithmic interpolation [8].

After an incubation over two hours with the virus-containing inoculum at 37° C., the cells were there-fold washed off from residual virus by centrifugation with 300 g over 10 min in the 30-fold volume of the medium and in every well of 24-wells plate were added 500 ml of the growth medium with corresponding AZT-concentration.

The mixed samples were cultivated in a thermostat at 37° C., 5% CO₂ and 80% moisture content.

As a result an examples of undergone and in-undergone to HBO-exposure cells which were contacted with different AZT-concentrations were obtained (10⁻¹²; 10⁻¹¹; 10⁻¹⁰; 10⁻⁹; 10⁻⁸; 10⁻⁷; 10⁻⁶ M).

The monitoring results of viability of oxygenated and not-oxygenated cells Supt1, inoculated HIV-1BRU with different AZT-concentrations, are shown in Table 1.

TABLE 1
Dynamics of the viability change of the cell culture Supt1 with
different AZT-concentrations, subjected and not subjected to the
HBO, corresponding with monitoring results of MTT-test in per cents.

| AZT- | Days after AZT addition | | | | | |
|---|---|---|---|---|---|---|
| concentration | 2 | | 3 | | 5 | |
| (M) | HBO− | HBO+ | HBO− | HBO+ | HBO− | HBO+ |
| 0 | 100 | 107.9 | 100 | 108.2 | 100 | 104.7 |
| 10⁻⁹ | 95.2 | 98.3 | 98.6 | 102.1 | 80.1 | 97.3 |
| 10⁻⁸ | 100.6 | 107.8 | 118.3 | 100.9 | 85.9 | 85.6 |
| 10⁻⁷ | 53.8 | 93.0 | 114.1 | 95.2 | 79.2 | 92.0 |
| 10⁻⁶ | 52.7 | 89.3 | 80.2 | 96.0 | 73.0 | 87.8 |
| 10⁻⁵ | 72.3 | 79.3 | 70.0 | 85.4 | 71.9 | 80.5 |
| 10⁻⁴ | 63.1 | 65.2 | 50.5 | 73.6 | 55.1 | 61.1 |
| 10⁻³ | 45.3 | 52.3 | 51.1 | 55.6 | 25.3 | 35.0 |

### EXAMPLE 2

### HBO as the Method of the Increase of AZT-efficiency in the Suppression of the AZT-sensitive HIV Strain Reproduction.

The experimental procedure was identically with that described in Example 1, except that there were monitored HIV-BRU antigen expression by the method of polyclonal EIA.

TABLE 2
Content of viral antigen in oxygenated (OX+) and not
oxygenated (OX−) supernatant cell culture Supt1, which
was infected with HIV-1_{BRU}, and different AZT concentrations.

| | 3(**) | | 5(**) | |
|---|---|---|---|---|
| [AZT] M | OX− | OX+ | OX− | OX+ |
| 0 | 28.2 | 16.9 | 100.0 | 83.4 |
| 10⁻¹¹ | 14.4 | 8.7 | 83.4 | 62.0 |
| 10⁻⁹ | 14.5 | 7.4 | 82.3 | 21.5 |
| 10⁻⁷ | 8.0 | 8.8 | 50.2 | 9.9 |

(**)-day after cells being infected

Comparing the amount of ED₅₀ for oxygenated and not oxygenated samples it can be seen, that AZT concentration, which is required for 50%-suppression of HIV, in oxygenated samples is lower by (10)^{3,4} that in not oxygenated samples (Table 3).

TABLE 3
ED₅₀(*) in Supt1/HIV-1_{BRU} for oxygenated (OX+)
and not oxygenated (OX−) samples.

| | OX− | OX+ |
|---|---|---|
| Ig ED₅₀ | −7.0 | −10.4 |

(*) ED₅₀ was derived on 5 day after infection.

The above data are attesting that the HBO increases anti-HIV efficiency of AZT.

### EXAMPLE 3

### HBO as the Method for Increase of the AZT-efficiency in the Reproduction Suppression of the AZT-resistant HIV-1_{A216} Strain.

The experimental procedure was identically with that described in Example 1, except that there were monitored HIV-1_{A216} antigen expression by the method of polyclonal EIA.

Influence of HBO and AZT on AZT-resistant HIV-1_{A216} strain is shown in Table 4.

TABLE 4
Content of viral antigen in oxygenated (OX+) and not oxygenated
(OX−) supernatant cell culture Supt1, which was infected with
HIV-1_{A216}, and different AZT concentrations.

| | 2(**) | | 3(**) | |
|---|---|---|---|---|
| [AZT] M | OX− | OX+ | OX− | OX+ |
| 0 | 55.8 | 48.4 | 100.0 | 65.6 |
| 10⁻¹¹ | 48.2 | 29.6 | 92.1 | 44.5 |
| 10⁻⁸ | 33.1 | 29.3 | 70.7 | 51.8 |
| 10⁻⁶ | 21.8 | 20.5 | 51.1 | 33.3 |

(**)-day after cells being infected

Comparing the amount of ED₅₀ for oxygenated and not oxygenated samples it can be seen, that AZT concentration, which is required for 50%-suppression of HIV, in oxygenated samples is lower by (10)² that in not oxygenated samples (Table 5).

TABLE 5
ED₅₀(*) in Supt1/HIV-1_{A216} for oxygenated (OX+)
and not oxygenated (OX−) samples.

| | OX− | OX+ |
|---|---|---|
| Ig ED₅₀ | −6.0 | −8.0 |

The above given data are attesting that the HBO increase anti-HIV efficiency of AZT to the resistance strain of the HIV.

Thus, the HBO can be considered as a consequence of the synergism of both above indicated effects.

The proposed method is, in principle, distinguishing from the existing methods in that said method is being non-cytotoxic and, moreover, is increasing anti-HIV AZT-efficiency to the AZT-resistant strain of a HIV, as well as to the AZT-sensible.

What is claimed is:

1. The use of the HBO as the method of the increase (enhancement) of the efficiency for the suppression of the Human Immunodeficiency Virus reproduction and the decrease of the cytotoxic effect too.
